(19) United States
(12) Patent Application Publication
(10) Pub. No.: US 2022/0214361 A1
(43) Pub. Date: Jul. 7, 2022

(54) Method for Screening Candidate Drug for Treating Degenerative Brain Diseases

(71) Applicant: THE INDUSTRY & ACADEMIC COOPERATION IN CHUNGNAM NATIONAL UNIVERSITY (IAC), Daejeon (KR)

(72) Inventor: Eunhee KIM, Daejeon (KR)

(21) Appl. No.: 17/608,970

(22) PCT Filed: Oct. 24, 2019
(86) PCT No.: PCT/KR2019/014071

§ 371 Date: Nov. 4, 2021

(30) Foreign Application Priority Data
May 21, 2019 (KR) 10-2019-0059724

(51) Int. Cl. G01N 33/68

(52) U.S. Cl. CPC G01N 33/6896; G01N 2500/10; G01N 2500/04

(57) ABSTRACT

In the present invention, it has been confirmed that FAF1 protein, which is reported to induce neuronal death, is secreted through exocytosis while being contained in exosomes, not through general protein transport pathways, thereby inducing apoptosis of other cells, and thus, through a process for selecting a test substance capable of suppressing the extracellular secretion of FAF1 protein, it is possible to screen for a therapeutic agent for degenerative brain diseases of which the major cause is neuronal death.

### BACKGROUND OF THE INVENTION

### 1. Field of the Invention

The present invention relates to a method for developing a therapeutic agent for degenerative brain disease by screening a substance capable of suppressing the extracellular secretion of FAF1 protein in the cells expressing FAF1 protein.

### 2. Description of the Related Art

Degenerative brain diseases are diseases that occur in the brain among degenerative diseases that develop with aging. These include Alzheimer's disease, Parkinson's disease, stroke, Huntington's disease, and spinal cord injury, and the number of patients with neurodegenerative diseases has increased rapidly over the past 20 years. Since degenerative brain diseases have a lack of research on the cause and treatment for these diseases, prevention is the most important. A common pathological phenomenon of degenerative brain disease is the death of central nerve cells. Unlike cells of other organs, central nerve cells are almost impossible to regenerate once apoptosis occurs, and thus permanent loss of function is inevitable. Until now, treatment methods for such degenerative brain diseases have been developed focusing on the analysis of the apoptosis mechanism of neurons themselves and suppression of apoptosis based thereon.

Parkinson's disease is a degenerative brain disease of the central nervous system caused by dopamine deficiency due to the death of dopaminergic neurons in the substantia nigra and corpus striatum in the midbrain. Since the exact cause of the selective death of dopaminergic neurons has not been elucidated so far, the development of effective therapeutic agents and diagnostic reagents is insufficient. Symptoms of Parkinson's disease include motor function related symptoms such as expressionless face, rigidity, tremor, bent posture, and bradykinesia. The number of Parkinson's disease (PD) patients in Korea was 61,565 as of 2010, and increasing at an average annual rate of 8.7%. This number was 85,888 in 2014, of which 95.7% were over 60 years old. The prevalence of the disease was correlated with the age of the patient, and the gender ratio was 33,831 males and 52,057 females.

Fas (Fas receptor, CD95, Apo1 or TNFRSF6) is one of death receptors, and Fas and Fas ligand (FasL) play important roles in apoptosis. When three Fas ligands bind to three Fas molecules, an adapter protein called FADD (Fas-associated death domain) binds to DD (death domain) of Fas. Thereafter, when DED (death effector domain) of inactive caspase-8 (FLICE or MACH) binds to DED (death effector domain) of FADD, caspase-8 is activated, and eventually apoptosis is induced by activating effector caspase that causes apoptosis. [Marsters, S. A. et al., Curr. Biol. 8:525-528, (1998)].

FAF1 (Fas-associated factor 1) is a protein known to have a function of inducing apoptosis by binding to Fas antigen, and its expression is known to significantly increase in the frontal cortex of patients with Parkinson's disease. In addition, it has been reported that inhibition of mitochondrial complex I, oxidative stress and increased alpha-synuclein expression in Parkinson's disease patients specifically increase the expression or FAF1. The increased expression of FAF1 induces apoptosis and enhances the toxic effects of stressors on Parkinson's disease, including oxidative stress, mitochondrial complex I inhibition, and proteasome inhibition [Keting Chu et al., Proc. Natl. Acad. Sci. USA, 92(25), pp. 11894-11898 (1995)]. As such, FAF1 is a protein that plays an important role in the pathogenesis and progression of Parkinson's disease.

So far, it is known that the expression of FAF1 is increased in Parkinson's disease patients and induces apoptosis by interacting with Fas receptor domain. However, it has not been revealed that FAF1 is secreted out of the cells, the exact secretion pathway, and that the secreted FAF1 induces apoptosis in adjacent cells.

While searching for a new therapeutic agent for Parkinson's disease, the present inventors have revealed that the FAF1 protein, which is known to be overexpressed in Parkinson's disease patients, is extracellularly secreted, identified a novel mechanism that the protein is secreted through a pathway other than the typical protein transport pathway, and revealed that the FAF1 protein secreted by this pathway induces apoptosis in adjacent cells. Based on the above mechanism, a substance capable of suppressing the extracellular secretion of FAF1 protein can be an effective treatment candidate for Parkinson's disease.

### SUMMARY OF THE INVENTION

It is an object of the present invention to provide a method for developing a therapeutic agent for degenerative brain disease by screening a substance capable of suppressing the extracellular secretion of FAF1 protein in the cells expressing FAF1 protein.

To achieve the above object, the present invention provides a method for screening a therapeutic agent for degenerative brain disease comprising the following steps:

1) treating a test substance to the cells expressing FAF1 (Fas associated factor 1) protein;

2) measuring the amount of extracellular secretion of FAF1 protein in the cells of step 1); and

3 selecting a test substance by which the amount of extracellular secretion of the FAF1 protein in step 2) is decreased compared to the control group not treated with the test substance.

### Advantageous Effect

In the present invention, it has been confirmed that FAF1 protein is secreted through exocytosis while being contained in exosomes, not through general protein transport pathways, thereby inducing apoptosis of other cells, and thus, through a process for selecting a test substance capable of suppressing the extracellular secretion of FAF1 protein, it is possible to screen for a therapeutic agent for degenerative brain diseases.

### BRIEF DESCRIPTION OF THE DRAWINGS

FIG. 1 is a diagram showing the results of Western blotting performed to confirm the extracellular secretion of FAF1 protein.

media: culture solution

WCL: whole cell lysate

FIG. 2a is a graph showing the relative secretion amount of FAF1 protein secreted into the culture medium to confirm whether the FAF1 protein is secreted through exocytosis.

FIG. 2b is a graph showing the relative secretion amount of FAF1 protein secreted into the culture medium in the group treated with the compound Brefeldin A (BFA) and the control group not treated to confirm whether the FAF1 protein is secreted through the endoplasmic reticulum-Golgi apparatus protein transport pathway.

FIG. 3 is a graph showing the relative secretion amount of FAF1 protein by treating a compound that increases or decreases the secretion of exosomes to confirm whether the FAF1 protein is secreted through the exosomes.

CMV vector: a group not-overexpressing FAF1

Flag FAF1: a group overexpressing FAF1

FIG. 4 is a diagram showing the results of Western blotting performed to confirm whether the FAF1 protein is secreted through the exosomes.

FIG. 5 is a diagram showing the results of Western blotting performed to confirm that the FAF1 protein secreted from the donor cells migrated to the recipient cells.

FIG. 6 is a set of graphs showing the results of measuring the degree of apoptosis using flow cytometry to confirm that the FAF1 protein secreted from the donor cells induced apoptosis of the recipient cells.

FIG. 7 is a set of photographs showing the results of observing the recipient cells using a confocal microscope to confirm that the FAF1 protein secreted from the donor cells migrated to the recipient cells.

GFP-vector: a group in which the medium of the donor cells not-expressing FAF1 protein was concentrated and treated to the recipient cells.

GFP-FAF1: a group in which the medium of the donor cells overexpressing GFP-conjugated FAF1 protein was concentrated and treated to the recipient cells.

### DESCRIPTION OF THE PREFERRED EMBODIMENTS

Hereinafter, the present invention is described in detail.

The present invention provides a method for screening a therapeutic agent for degenerative brain disease comprising the following steps:

1) treating a test substance to the cells expressing FAF1 (Fas associated factor 1) protein;

2) measuring the amount of extracellular secretion of FAF1 protein in the cells of step 1); and

3) selecting a test substance by which the amount of extracellular secretion of the FAF1 protein in step 2) is decreased compared to the control group not treated with the test substance.

The FAF1 protein is a protein that binds to the intracytoplasmic domain of Fas known to be involved in apoptosis, and is one of the representative proteins that bind to Fav without DD. Human FAF1 protein is located in the cytoplasm as a Fas-binding protein, and although its function in the nucleus has not been clearly elucidated, the possibility that it exists in the nucleus by its NLS domain is emerging. In addition, the possibility of modification of FAF1 by the phosphorylation by protein kinase CK2, glycosylation, and the like is suggested. In fact, FAF1 is known to have two UB domains, one X domain, and one UX domain, which are domains seen in proteins or the ubiquitination pathway. Among them, the UX domain is known as a domain involved in the mechanism related to ubiquitin. In FAF1, the 289^{th} and 291^{st} amino acids are phosphorylated by protein kinase CK2 holoenzyme or CD2alpha subunit.

In the method above, the degenerative brain disease can be Parkinson's disease, but not always limited thereto.

In the method above, the test substance of step 1) can be any one selected from the group consisting of peptides, proteins, non-peptidic compounds, synthetic compounds, fermentation products, cell extracts, plant extracts, and animal tissue extracts, but not always limited thereto.

The test substance can be a single compound, a mixture of compounds (eg, a natural extract or a cell or tissue culture), an antibody or a peptide, or can be obtained from a library of synthetic or natural compounds. Methods for obtaining libraries of such compounds are known in the art. Libraries of synthetic compounds are commercially available from Maybridge Chemical Co. (UK), Comgenex (USA), Brandon Associates (USA), Microsource (USA) and SigmaAldrich (USA), and libraries of natural compounds are commercially available from Pan Laboratories (USA) and MycoSearch (USA).

In the method above, the FAF1 protein of step 1) may be secreted by exocytosis.

The exocytosis is an energy-consuming process, which is emiocytosis in which substances in cells are discharged to the outside of the cells. At this time, the plasma membranes fall out of the cells.

In the method above, the FAF1 protein of step 1) may be secreted into exosomes.

Exosomes are small, membrane-structured vesicles secreted from various types of cells. The diameter of the exosomes is reported to be approximately 30˜100 nm. In the study using an electron microscope, it was observed that exosomes originate in specific compartments within cells called multivesicular bodies (MVBs), rather than directly detach from the plasma membrane, and are released and secreted out of the cells. That is, after the multivesicular bodies are fused to the plasma membrane, vesicles are released into the extracellular environment, which are called exosomes.

In the method above, the FAF1 protein of step 1) may induce apoptosis of other cells.

The apoptosis is a type of programmed cell death seen in multicellular organisms. Apoptosis is defined as the death of a cell due to cell morphology and biochemical changes inside the cell. This process ends with cell swelling and cracking, cell membrane changes, chromatin condensation and chromosome cleavage, and the cell being eaten and processed by other cells. Apoptosis is non-harmful to the organism and beneficial to the life cycle, in contrast to necrosis, a cell death caused by sudden cell damage. The formation of fingers and toes during the differentiation process of a human embryo is a representative example of apoptosis. In addition, apoptosis is an important mechanism for cell replacement, tissue remodeling, and clearance of injured cells. However, if apoptosis is incorrectly controlled, various diseases such as cancer, autoimmune diseases, neurodegenerative disorders, and viral infections can be induced.

Depending on the location of the inducing factor, apoptosis is divided into two cases: Intrinsic apoptosis and extrinsic apoptosis. Intrinsic apoptosis is activated by the intracellular signals released when cells are under stress and the proteins released from the mitochondrial intermembrane space. Mitochondria are very important organelles for living things. Without mitochondria, cells stop respiration and rapidly die. Apoptotic protein that targets mitochondria affects mitochondria in a variety of ways. This protein expands mitochondria by making a membrane pore on the mitochondrial membrane, or increases the permeability of the mitochondrial membrane so that apoptotic substances in the mitochondrial intermembrane space are released. In addition, nitric oxide (NO) reduces the concentration gradient formed in mitochondria, increases the permeability of the mitochondrial membrane, and induces apoptosis.

Extrinsic apoptosis has several specificities different from intrinsic apoptosis. In this case, apoptosis can be induced without the effect of mitochondria, and it can be called apoptosis induced by immune cells, peripheral cells, or cytokines that are expressed by themselves. Therefore, factors different from the former case are involved, and dozens of receptor families are found because a cell must recognize a specific cytokine.

In the method above, the secretion amount in step 2) can be measured by any one method selected from the group consisting of Western blotting, immunoprecipitation assay, dual luciferase reporter assay, enzyme-linked immunosorbent assay (ELISA), and immunohistochemistry, but not always limited thereto.

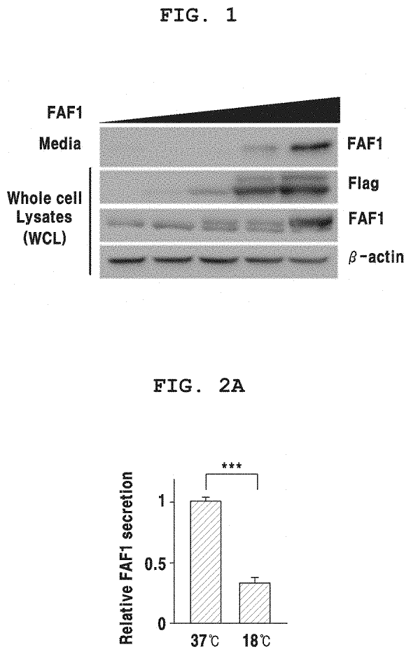

In a specific embodiment of the present invention, the present inventors overexpressed FAF1 by transducing the pcDNA 3.1-3x Flag tag-FAF1 expression vector into SH-SY5Y cells to confirm whether the FAF1 protein was extracellularly secreted. Then, by analyzing the protein in the culture medium by Western blotting, it was confirmed that the FAF1 protein was extracellularly secreted (FIG. 1).

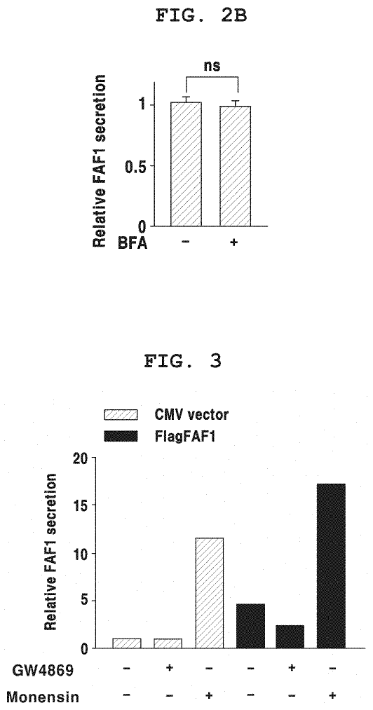

In addition, the present inventors measured the expression level of the FAF1 protein secreted in cells overexpressing FAF1 by Western blotting under two temperature conditions: 18° C., a temperature that inhibits exocytosis, and 37° C., an in vivo temperature. As a result, it was confirmed that the secretion of FAF1 protein was inhibited at 18° C., and that FAF1 was secreted through exocytosis (FIG. 2a).

In addition, the cells overexpressing FAF1 were treated with Brefeldin A (BFA), a compound that inhibits protein transport in the ER-Golgi pathway, and the expression level of the FAF1 protein was measured by Western blotting. As a result, it was confirmed that the expression level of the FAF1 protein was constant regardless of the treatment of BFA, and thus it was confirmed that the FAF1 protein was not secreted through a typical protein transport pathway (FIG. 2b).

In addition, the cells overexpressing FAF1 were treated with two compounds that increased or decreased the secretion of exosomes, and the expression level of the FAF1 protein was measured. As a result, the secretion amount of the FAF1 protein was increased when a substance that increased exosome secretion was treated, and when a substance that decreased exosome secretion was treated, the secretion amount of the FAF1 protein was decreased. Therefore, it was confirmed that the secretion amount of the FAF1 protein was regulated according to the secretion of exosomes (FIG. 3).

In addition, exosomes were isolated from the culture medium in which the cells overexpressing FAF1 were cultured, and the exosomes were measured by Western blotting. As a result, by confirming the presence of the FAF1 protein, it was confirmed that the FAF1 protein was secreted through exosomes (FIG. 4).

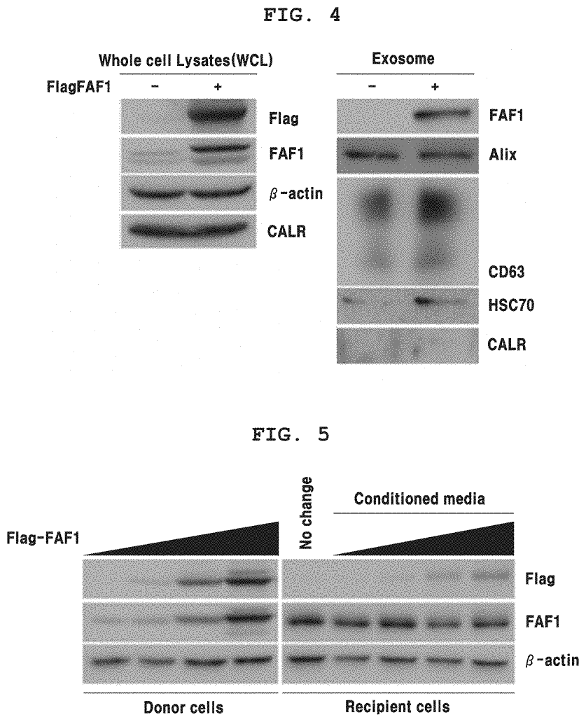

In addition, in order to confirm whether the FAF1 protein was transferred from the donor cells overexpressing FAF1 to the recipient cells, the medium of the donor cells was concentrated and treated to the recipient cells, followed by Western blotting. As a result, by confirming the presence of the flag tag-conjugated FAF1 protein, it was confirmed that the FAF1 protein secreted from the donor cells moved into the recipient cells (FIG. 5).

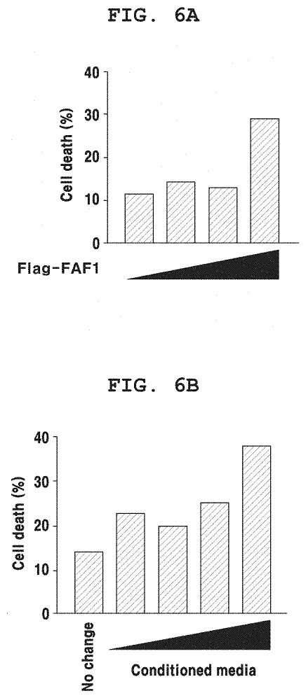

In addition, to confirm whether the FAF1 protein secreted from the donor cells caused apoptosis of the recipient cells, the recipient cells treated with the concentrated medium of the donor cells were analyzed by flow cytometry. As a result, it was confirmed that as the donor cells overexpressed FAF1, the proportion of apoptotic cells was increased, thereby confirming that the FAF1 protein secreted from the donor cells induced apoptosis of the recipient cells (FIG. 6).

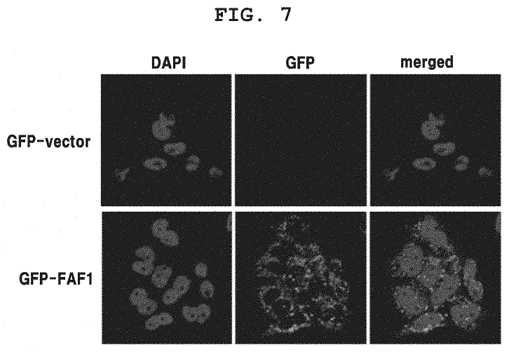

In addition, in order to confirm whether the FAF1 protein secreted from the donor cells migrated into the recipient cells, the recipient cells were treated with the concentrated medium of the donor cells and observed with a confocal microscope. As a result, by confirming that the FAF1 protein labeled with GFP (Green fluorescent protein) was observed, it was confirmed that the FAF1 protein secreted from the donor cells moved into the recipient cells (FIG. 7).

Therefore, it has been confirmed that the FAF1 protein, which has been reported to induce neuronal cell death, is secreted through exocytosis while being contained in exosomes, not through general protein transport pathways, thereby inducing apoptosis of other cells, and thus, through a process for selecting a test substance capable of suppressing the extracellular secretion of FAF1 protein, it is possible to screen for a therapeutic agent for degenerative brain diseases represented by Parkinson's disease.

Hereinafter, the present invention will be described in detail by the following examples and experimental examples.

However, the following examples and experimental examples are only for illustrating the present invention, and the contents of the present invention are not limited thereto.

### EXPERIMENTAL EXAMPLE 1: CONFIRMATION OF EXTRACELLULAR SECRETION OF FAF1 PROTEIN

Human neuroblastoma SH-SY5Y cells were transfected with an expression vector in which the FAF1 gene was introduced to confirm whether the FAF1 Protein was extracellularly secreted.

Human neuroblastoma SH-SY5Y cells were aliquoted into 60 mm culture dishes at the density of 5×10⁵ cells/dish, and subcultured in DMEM (Dulbecco/s Modified Eagle's Medium) supplemented with antibiotics and 10% FBS (fetal bovine serum) under the conditions of 37° C. and 5% CO₂ for 24 hours. 0, 0.25, 0.5, 1 and 2 μg of the expression vector to be introduced into cells and BioT, 1.5 times the amount of each expression vector, were mixed with 200 [symbol] of serum-free DMEM (Dulbecco/s Modified Eagle's Medium), followed by reaction at room temperature for 5 minutes. The cultured SH-SY5Y cells were transfected with the mixture and cultured for 24 hours, then the medium was removed and washed with PBS buffer. Thereafter, the medium was replaced with serum-free DMEM (Dulbecco/s Modified Eagle's Medium) and the cells were further cultured for 24 hours. The medium was transferred to a 15 ml tube and centrifuged at 1500 rpm for 2 minutes to obtain a supernatant. The obtained supernatant was placed in Amicon Ultra-4 Centrifugal Filter, and centrifuged again at 4000×g, 4° C. for 15 minutes. The protein filtered by the filter was analyzed by Western blotting.

As a result, as shown in FIG. 1, the expression level of the FAF1 protein was increased as the amount of the expression vector transduced into the cells was increased. These results suggest the possibility that the FAF1 protein was secreted into the culture medium (FIG. 1).

### EXPERIMENTAL EXAMPLE 2: SEARCHING SECRETORY PATHWAY OF FAF1 PROTEIN

### <2-1> Confirmation Whether FAF1 Protein is Secreted by Exocytosis

It was confirmed whether the FAF1 protein, which was confirmed to be present in the medium in Experimental Example 1, was secreted through the endogenous regulatory mechanism of cells, that is, exocytosis, not due to cell damage or apoptosis.

Particularly, SH-SY5Y cells were aliquoted into 60 mm culture dishes at the density of 5×10⁵ cells/dish, and subcultured in DMEM (Dulbecco/s Modified Eagle's Medium) supplemented with antibiotics and 10% FBS (fetal bovine serum) under the conditions of 37° C. and 5% CO₂ for 24 hours. The cultured SH-SY5Y cells were transfected with 2 μg of 3x Flag tag-FAF1 expression vector to overexpress FAF1 protein, cultured for 24 hours, and then washed with PBS buffer. After replacing the medium with DMEM (Dulbecco/s Modified Eagle's Medium), the cells were further cultured in an incubator at 18° C. and 37° C. for 24 hours. The medium was transferred to a 15 ml tube and centrifuged at 1500 rpm for 2 minutes to obtain a supernatant. The obtained supernatant was placed in Amicon Ultra-4 Centrifugal Filter, and centrifuged again at 4000×g, 4° C. for 15 minutes. The protein filtered by the filter was analyzed by Western blotting.

As a result, as shown in FIG. 2a, the secretion amount of FAF1 from the cells cultured at 18° C., a temperature at which exocytosis is inhibited, was significantly decreased compared to the cells cultured at 37° C. (FIG. 2a). These results suggest that the FAF1 protein was secreted through exocytosis.

### <2-2> Confirmation of Secretory Pathway of FAF1 Protein

The following experiment was performed to confirm whether the FAF1 protein is secreted through the ER-Golgi pathway, which is a typical protein secretion pathway, or an atypical pathway.

Particularly, SH-SY5Y cells were aliquoted into 60 mm culture dishes at the density of 5×10⁵ cells/dish, and subcultured in DMEM (Dulbecco/s Modified Eagle's Medium) supplemented with antibiotics and 10% FBS (fetal bovine serum) under the conditions of 37° C. and 5% CO₂ for 24 hours. The cultured SH-SY5Y cells were transfected with 2 μg of 3x Flag tag-FAF1 expression vector to overexpress FAF1 protein, cultured for 24 hours, and then washed with PPS buffer. Thereafter, the medium was replaced with serum-free DMEM (Dulbecco/s Modified Eagle's Medium), and the cells were treated with 10 μM Brefeldin A (BFA), a compound that inhibits protein secretion through the ER-Golgi pathway, and further cultured in an incubator for 24 hours. The medium was transferred to a 15 ml tube and centrifuged at 1500 rpm for 2 minutes to obtain a supernatant. The obtained supernatant was placed in Amicon Ultra-4 Centrifugal Filter, and centrifuged again at 4000×g, 4° C. for 15 minutes. The protein filtered by the filter was analyzed by Western blotting.

As a result, as shown in FIG. 2b, there was no difference in the expression level of FAF1 protein between the group treated with Brefeldin A (BFA) and the group not treated with BFA. Therefore, it was confirmed that the FAF1 protein was secreted through a pathway other than the ER-Golgi pathway, a typical protein secretion pathway (FIG. 2b).

### <2-3> Confirmation of Whether FAF1 Protein is Secreted Through Exosomes

Exosomes are nano-sized extracellular vesicles that serve as information messengers between cells in vivo. The following experiment was performed to confirm whether the FAF1 protein was secreted through exosomes.

Particularly, SH-SY5Y cells were aliquoted into 60 mm culture dishes at the density of 5×10⁵ cells/dish, and subcultured in DMEM (Dulbecco/s Modified Eagle's Medium) supplemented with antibiotics and 10% FBS (fetal bovine serum) under the conditions of 37° C. and 5% CO₂ for 24 hours. The cultured SH-SY5Y cells were transfected with 2 μg of 3x Flag tag-FAF1 expression vector to overexpress FAF1 protein, cultured for 24 hours, and then washed with PBS buffer. Thereafter, the medium was replaced with serum-free DMEM (Dulbecco/s Modified Eagle's Medium), and the cells were treated with 10 μM Monensin compound to increase exosome secretion and 10 μM GW4869 compound to decrease exosome secretion, and further cultured for 24 hours. The medium was transferred to a 15 ml tube and centrifuged at 1500 rpm for 2 minutes to obtain a supernatant. The obtained supernatant was placed in Amicon Ultra-4 Centrifugal Filter, and centrifuged again at 4000×g, 4° C. for 15 minutes. The protein filtered by the filter was analyzed by Western blotting.

As a result, as shown in FIG. 3, the secretion amount of FAF1 in the group treated with GW4869 compound was decreased compared to that in the control group, and the secretion amount of FAF1 in the group treated with Monensin compound was significantly increased compared to that in the control group. Therefore, it was confirmed that the FAF1 protein was secreted through exosomes (FIG. 3).

### <2-4> Confirmation of FAF1 Protein in Exosomes Through Exosome Isolation

In order to confirm the presence of FAF1 protein in the extracellularly secreted exosomes, the following experiment was performed.

Particularly, SH-SY5Y cells were subcultured in DMEM (Dulbecco/s Modified Eagle's Medium) supplemented with antibiotics and 10% FBS (fetal bovine serum) until they occupied about 70% of the area in 150 mm culture dishes under the conditions of 37° C. and 5% CO₂ for 24 hours. The cultured SH-SY5Y cells were transfected with 7 μg of 3x Flag tag-FAF1 expression vector to overexpress FAF1 protein, cultured for 24 hours, and then washed with PBS buffer. Thereafter, the medium was replaced with DMEM (Dulbecco/s Modified Eagle's Medium) from which exosomes derived from FBS were removed, and the cells were further cultured for 48 hours. The medium was transferred to a 15 ml tube and centrifuged at 2000×g for 20 minutes to obtain a supernatant. The obtained supernatant was centrifuged at 10000×g for 30 minutes to obtain a supernatant, and then centrifuged again at 140000×g for 70 minutes. After discarding the supernatant, 1 ml of PBS was added and resuspended to fill ¾ of the tube, and centrifuged at 140000×g for 70 minutes. The supernatant was discarded. After adding 80 [symbol] of RIPA buffer to the precipitate and resuspending thereof, it was transferred to an EP tube. The precipitate in the EP tube was analyzed by Western blotting.

As a result, as shown in FIG. 4, FAF1 protein was not detected in exosomes of the cells not overexpressing FAF1, but FAF1 protein was detected in exosomes of the cells overexpressing FAF1. Therefore, it was confirmed that FAF1 was secreted through exosomes (FIG. 4).

### EXPERIMENTAL EXAMPLE 3: CONFIRMATION OF EFFECT OF FAF1 PROTEIN SECRETED FROM DONOR CELLS ON RECIPIENT CELLS

### <3-1> Enrichment of Donor Cell Medium

To confirm the effect of the FAF1 protein extracellularly secreted from the donor cells on the recipient cells, the donor cell medium was enriched and the FAF1 protein in the medium was analyzed.

Particularly, SH-SY5Y cells were aliquoted into 60 mm culture dishes at the density of 5×10⁵ cells/dish, and subcultured in DMEM (Dulbecco/s Modified Eagle's Medium) supplemented with antibiotics and 10% FBS (fetal bovine serum) under the conditions of 37° C. and 5% CO₂ for 12 hours. 0, 1, 2 and 4 μg of the 3x Flag tag-FAF1 expression vector to be introduced into cells and BioT, 1.5 times the amount of each expression vector, were mixed with 200 [symbol] of serum-free DMEM (Dulbecco/s Modified Eagle's Medium), followed by reaction at room temperature for 5 minutes. The cultured SH-SY5Y cells were transfected with the mixture and cultured for 24 hours, then the medium was removed and washed with PBS buffer. Thereafter, the medium was replaced with serum-free DMEM (Dulbecco/s Modified Eagle's Medium) and the cells were further cultured for 24 hours. The medium was transferred to a 15 ml tube and centrifuged at 1500 rpm for 2 minutes to obtain a supernatant. The obtained supernatant was placed in Amicon Ultra-4 Centrifugal Filter, and centrifuged again at 4000×g, 4° C. for 15 minutes. The protein filtered by the filter was transferred to an EP tube to obtain the enriched conditioned medium. The donor cells present in the culture dish were subjected to Western blotting (FIG. 5).

As a result, as shown in FIG. 5, the expression of FAF1 protein in the donor cells was increased as the amount of the expression vector introduced into the cells was increased (FIG. 5).

### <3-2> Confirmation of Whether FAF1 Protein Secreted from Donor Cells Migrates to Recipient Cells and Causes Apoptosis

In order to confirm whether the FAF1 protein extracellularly secreted from the donor cells migrates to the recipient cells and induces apoptosis, the recipient cells were treated with the enriched donor cell medium obtained in Experimental Example 3-1, and the following experiment was performed.

Particularly, SH-SY5Y cells were aliquoted into 60 mm culture dishes at the density of 5×10⁵ cells/dish, and subcultured in DMEM (Dulbecco/s Modified Eagle's Medium) supplemented with antibiotics and 10% FBS (fetal bovine serum) under the conditions of 37° C. and 5% CO₂ for 12 hours. The cultured cells were treated with the enriched donor cell medium obtained in Experimental Example 3-1 for 48 hours. Then, cells were harvested and Western blotting was performed with some cells (FIG. 5). Some cells were placed in an EP tube containing 1 ml of PBS buffer, to which 100 [symbol] of 50 μg/ml propidium iodide (PI) was added, and the degree of apoptosis was measured by flow cytometry (FACS) (FIG. 6).

As a result, as shown in FIG. 5, the expression of the Flag tag was increased in the recipient cells in proportion to the amount of the expression vector treated to the donor cells. Therefore, it was confirmed that the FAF1 protein secreted from the donor cells migrated into the recipient cells.

In addition, as shown in FIG. 6, apoptosis was induced in the donor cells proportional to the amount of expression vector treated to the donor cells, and apoptosis of recipient cells was induced depending on the amount of the enriched donor cell medium treated (FIG. 6).

### <3-3> Confirmation or Whether FAF1 Protein Extracellularly Secreted from Donor Cells Moves into Recipient Cells

In order to confirm whether the FAF1 protein extracellularly secreted from the donor cells moves into the recipient cells, the cells were stained and observed with a confocal microscope.

Particularly, SH-SY5Y cells were subcultured in DMEM (Dulbecco/s Modified Eagle's Medium) supplemented with antibiotics and 10% FBS (fetal bovine serum) until they occupied about 70% of the area in 150 mm culture dishes under the conditions of 37° C. and 5% CO₂ for 24 hours. 0, 1, 2 and 4 μg of the pEGFP-GFP-FAF1 expression vector to be introduced into cells and BioT, 1.5 times the amount of each expression vector, were mixed with 200 [symbol] of serum-free DMEM (Dulbecco/s Modified Eagle's Medium), followed by reaction at room temperature for 5 minutes. The cultured SH-SY5Y cells were transfected with the mixture, cultured for 24 hours, and then washed with PBS buffer. Thereafter, the medium was replaced with serum-free DMEM (Dulbecco/s Modified Eagle's Medium), and the cells were further cultured for 24 hours. The medium was transferred to a 15 ml tube and centrifuged at 1500 rpm for 2 minutes to obtain a supernatant. The obtained supernatant was placed in Amicon Ultra-4 Centrifugal Filter, and centrifuged again at 4000×g, 4° C. for 15 minutes. The protein filtered by the filter was transferred to an EP tube to obtain the enriched donor cell medium.

Recipient cells were aliquoted on 18 mm×18 mm cover slips coated with Poly-D-lysine at the density of 6×10⁴ cells/slip, and cultured for 24 hours. Then, the enriched donor cell medium was treated to the recipient cells, followed by further culture for 24 hours. Thereafter, the cells were fixed with 4% paraformaldehyde (PFA) at room temperature for 15 minutes, and the cell nuclei were stained with 4′6-diamidino-2-phenylindole (DAPI) for 10 minutes. After washing the cover slip 3 times with PBS for 10 minutes, the 1,4-diazabicyclo[2.2.2]octane (DABCO)-treated cover glass was covered on the slide glass and observed with a confocal microscope.

As a result, as shown in FIG. 7, the FAF1 protein labeled with GFP was observed in the recipient cells. Therefore, it was confirmed that the FAF1 protein extracellularly secreted from the donor cells migrated into the recipient cells (FIG. 7).

1. A method for screening a therapeutic agent for degenerative brain disease comprising the following steps:
1) treating a test substance to the cells expressing FAF1 (Fas associated factor 1) protein;
2) measuring the amount of extracellular secretion of FAF1 protein in the cells of step 1); and
3) selecting a test substance by which the amount of extracellular secretion of the FAF1 protein in step 2) is decreased compared to the control group not treated with the test substance.

2. The method for screening a therapeutic agent for degenerative brain disease according to claim 1, wherein the degenerative brain disease is Parkinson's disease.

3. The method for screening a therapeutic agent for degenerative brain disease according to claim 1, wherein the test substance in step 1) is any one selected from the group consisting of peptides, proteins, non-peptidic compounds, synthetic compounds, fermentation products, cell extracts, plant extracts, and animal tissue extracts.

4. The method for screening a therapeutic agent for degenerative brain disease according to claim 1, wherein the FAF1 protein is extracellularly secreted through exocytosis.

5. The method for screening a therapeutic agent for degenerative brain disease according to claim 1, wherein the FAF1 protein is extracellularly secreted through exosomes.

6. The method for screening a therapeutic agent for degenerative brain disease according to claim 4, wherein the extracellularly secreted FAF1 protein induces apoptosis in other cells.

7. The method for screening a therapeutic agent for degenerative brain disease according to claim 1, wherein the secretion amount in step 2) is measured by any one method selected from the group consisting of Western blotting, immunoprecipitation assay, dual luciferase reporter assay, enzyme-linked immunosorbent assay (ELISA), and immunohistochemistry

8. The method for screening a therapeutic agent for degenerative brain disease according to claim 5, wherein the extracellularly secreted FAF1 protein induces apoptosis in other cells.
